(19) United States
(12) Patent Application Publication
(10) Pub. No.: US 2008/0201954 A1
(43) Pub. Date: Aug. 28, 2008

(54) Apparatus for Shaving a Person's Hair by Way of Laser Radiation

(75) Inventor: Jens Meinschien, Dortmund (DE)

Correspondence Address: LERNER GREENBERG STEMER LLP, P O BOX 2480, HOLLYWOOD, FL, 33022-2480, US

(73) Assignee: LIMO PATENTVERWALTUNG GMBH & CO. KG, Gerstengrund (DE)

(21) Appl. No.: 12/051,447

(22) Filed: Mar. 19, 2008

Related U.S. Application Data
(63) Continuation of application No. PCT/EP2005/010079, filed on Sep. 19, 2005.

(51) Int. Cl. B26B 19/00; A61B 18/18

(52) U.S. Cl. 30/34.05; 606/9

(57) ABSTRACT

An apparatus for shaving a person's hair by way of laser radiation includes a base unit with at least one laser light source, a portable unit which a user can move to the region of the hair to be cut, and also a transmission device with a plurality of optical fibers. The fibers transmit the laser radiation emitted by the at least one laser light source from the base unit to the portable unit. The ends of the optical fibers are arranged in the portable unit in such a way that the laser radiation emitted from these ends can at least partially overlap and provide for a linear beam cross section in the overlapped state.

### CROSS-REFERENCE TO RELATED APPLICATIONS

This is a continuing application, under 35 U.S.C. § 120, of copending international application PCT/EP2005/010079, filed Sep. 19, 2005, which designated the United States; the prior application is herewith incorporated by reference in its entirety.

### BACKGROUND OF THE INVENTION

### Field of the Invention

The present invention relates to an apparatus for removing a person's hair by laser radiation. The laser apparatus has a base unit with at least one laser light source and a portable unit that can be moved by a user into the area of the hair to be cut off. A transmission device has at least one optical fiber which can transmit the laser radiation emanating from the at least one laser light source from the base unit to the portable unit.

An apparatus of the above-mentioned type is described in international publication WO 93/05920 A1. That apparatus, described as a hair ablation system, may comprise a base station and a portable hand piece that can be guided by the user into the work area in which the hair to be removed is located. The base station and the hand piece can be connected to one another by a cable which comprises an optical fiber. A laser light source, the light of which passes through a series of lenses before entering the optical fiber, is housed in the base station. After the laser radiation has been emitted from the optical fiber in the hand piece, it also passes through a plurality of lenses, in particular cylinder lenses, so that laser radiation having a linear cross section can be emitted from the hand piece for shaving human hair.

The disadvantage of such an apparatus is the complexity of the optical construction, which entails high production costs.

### BRIEF SUMMARY OF THE INVENTION

It is accordingly an object of the invention to provide a hair removal laser apparatus, which overcomes the above-mentioned disadvantages of the heretofore-known devices and methods of this general type and which provides for an apparatus that can be produced more cost effectively.

With the foregoing and other objects in view there is provided, in accordance with the invention, a laser shaving apparatus, comprising:

a base unit having at least one laser light source generating laser radiation;

a portable unit movable by a user into an area of hair to be cut;

a transmission device connected between said base unit and said portable unit for transmitting the laser radiation from said at least one laser light source of said base unit to said portable unit, said transmission device including a plurality of optical fibers for transmitting the laser radiation; and

said optical fibers having ends disposed in said portable unit, said ends being arranged to cause the laser radiation emitted therefrom to overlap at least partially and to form an elongated beam cross section in an overlapped state.

By providing a plurality of optical fibers for transmitting the laser radiation, the ends of which are arranged in the portable unit such that the laser radiation emitted from these ends can at least partially overlap and has an elongated beam cross section in the overlapped state, a beam cross section suitable for shaving can be achieved with simple means. In particular, in this way it is possible to achieve the elongated beam cross section suitable for shaving without additional optical means such as lenses, or with considerably less or more cost-effective optical means. In the case of an embodiment without lenses or the like in front of the emitting ends of the optical fibers, very precise and thus expensive mechanical holders can also additionally be dispensed with. Furthermore, the complex adjustment of such lenses and holders is no longer necessary during the production of the apparatus. An additional advantage is provided by the fact that the apparatus can continue to be used even in the case of defects in individual optical waveguides, since laser radiation is still transmitted by the other optical waveguides.

In particular, for this purpose, the ends of the optical fibers can be arranged substantially in a row next to one another in the portable unit, so that, in the overlapped state of the laser radiation, this results in a substantially linear beam cross section. This organization of the plurality of optical fibers thus enables the creation of a linear beam cross section without additional cylinder lenses or the like. This arrangement of the optical fibers can be created entirely passively during production, that is to say without laser operation, so that the production costs can be lowered further.

It is possible that the apparatus comprises a plurality of laser light sources, which are preferably formed as individual laser diodes or individual emitters of a laser diode bar. In particular, in this case, each laser light source can be assigned exactly one optical fiber. Compared to laser diode bars, individual laser diodes advantageously have a longer life expectancy and can be operated at higher temperatures, so that the cooling system has to meet lower requirements. Furthermore, independent operation of the individual laser diodes can be chosen, so that the failure of an individual laser diode does not lead to defects of further laser diodes and the apparatus can still be used anyhow. By way of example, this can be ensured by a series connection of the laser diodes with a fail safe feature such as a low-impedance bypass in the case of failure of a diode. Furthermore, the use of many identical or similar components, such as the use of a plurality of similar laser diodes and a plurality of similar optical fibers, contributes to the reduction of costs due to mass production.

An advantageous embodiment can result from respectively one optical fiber being able to be arranged in front of one of the laser light sources such that the laser light emitted from the laser light source passes directly into the optical fiber, in particular without previously passing through optical means, such as lenses or the like. Thus lenses can also be dispensed with on the entry side of the optical fibers, so that the costs can be lowered further. At most, a fast axis collimation lens could be arranged between a laser diode or laser diode bar and the optical fiber or optical fibers, in order to collimate the laser radiation to a large extent with regard to the large divergence in the so-called fast axis.

It is possible that the transmission means comprise a flexible cable, in which the optical fibers are densely packed. In this case, this results in a particularly dense and compact packing if the number of optical fibers is 7 or 19 or 37.

It is furthermore possible that the flexible cable comprises an electrical signaling line and/or at least one optical waveguide for guiding visible pilot radiation. By means of the electrical signaling line, the laser light source can be switched, for example. The pilot radiation can emanate from a light-emitting diode or laser diode which is suitable for generating visible light and can be fed to the portable unit by an additional optical fiber. By way of example, in the case that the laser radiation is emitted from the portable unit into a work area for shaving hair, the pilot radiation can assist the user in the targeted guidance of the laser radiation.

It is possible to use an inventive apparatus for disinfection purposes or for processing plastics. In this case, the output power of the at least one laser light source must of course be matched to the application. However, it is shown that the linear beam cross section generated by the appropriate arrangement of the optical fibers allows a welding, cutting or modification of plastics.

Other features which are considered as characteristic for the invention are set forth in the appended claims.

Although the invention is illustrated and described herein as embodied in apparatus for ablating a person's hair by laser radiation, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a schematic perspective view of the basic layout of the apparatus according to the invention;

FIG. 2 is a perspective view schematically illustrating the construction of an exemplary embodiment of a base unit of the novel apparatus;

FIG. 3 is a cross section through an exemplary embodiment of a holder of the optical fibers in the portable unit of the novel apparatus;

FIG. 4A is a cross section through an exemplary embodiment of a bundle of seven optical fibers of the apparatus;

FIG. 4B is a cross section through an exemplary embodiment of a bundle of 19 optical fibers of the apparatus;

FIG. 4C is a cross section through an exemplary embodiment of a bundle of 37 optical fibers of the apparatus;

FIG. 5A is a diagram of a two-dimensional intensity distribution in an application plane of the laser radiation emanating from the apparatus;

FIG. 5B is an intensity graph showing a one-dimensional representation of the intensity distribution according to FIG. 5A; and

FIG. 5C is a further one-dimensional representation of the intensity distribution according to FIG. 5A.

### DETAILED DESCRIPTION OF THE INVENTION

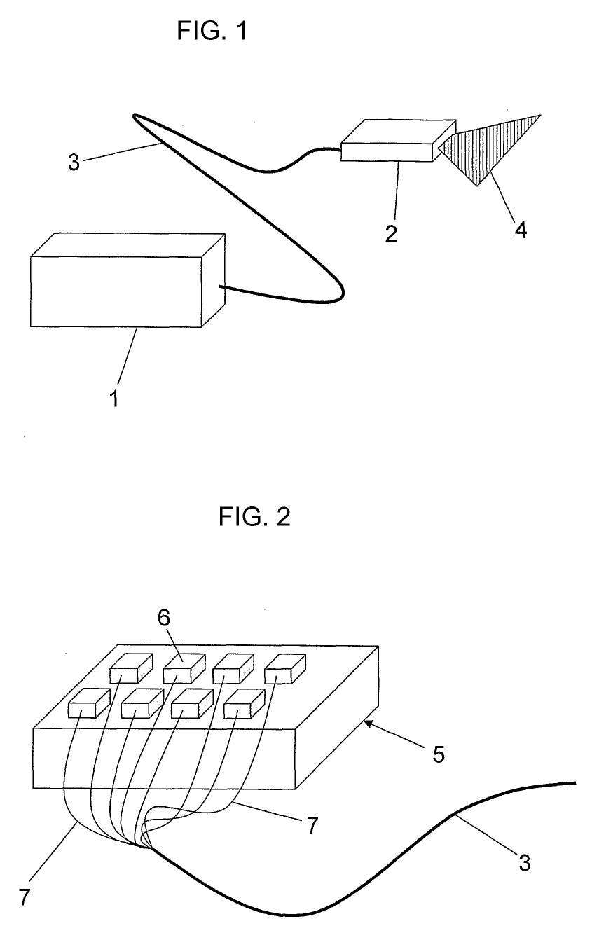

Referring now to the figures of the drawing in detail and first, particularly, to FIG. 1 thereof, the apparatus according to the invention comprises a base unit 1, a portable unit 2, and a flexible cable 3 connecting the base and portable units. The portable unit 2 may be formed with a slit-like opening on one side, through which laser radiation 4 can be emitted from the portable unit.

FIG. 2 schematically shows details of an exemplary embodiment of a base unit 1. The base unit 1 comprises a heat sink 5, to which a plurality of laser diodes 6 are attached. By arranging the laser diodes 6 on a common heat sink 5, the heat generated by the individual laser diodes 6 is distributed comparatively evenly. As a result, the thermal load on the laser diodes 6 is low on account of small temperature gradients.

By way of example, approximately 10 to 30 laser diodes 6, preferably approximately 19 laser diodes 6, can be provided. The laser diodes 6 can in each case have an optical power of approximately 3 W to 8 W at an emission wavelength between 800 nm and 1000 nm.

In an alternative embodiment, a laser diode bar with a plurality of emission sources can also be provided in place of a plurality of laser diodes 6. It is also possible to provide a plurality of laser diode bars.

An optical fiber 7, into which the light emanating from the appropriate laser diode 6 can enter, is positioned in front of each laser diode 6. Here, in the embodiment shown, no optical means such as lenses or the like are arranged between the laser diode 6 and that end of the optical fiber 7 at which the light enters. In the case of an appropriate distance and suitable positioning it can nevertheless be ensured that a large part of the light emitted by the laser diode is injected into the optical fiber 7. In each case the optical fibers 7 can have a core diameter of 100 μm and a numerical aperture of 0.22. The optical fibers 7 can be coated with metal to make them more flexible and to increase their breaking strength compared to uncoated optical fibers.

A lens, for example a fast axis collimation lens, can also be provided between the laser diode 6 and the appropriate optical fiber 7, in order to at least partially collimate the divergence of the laser light emitted by the laser diode 6 with regard to the direction perpendicular to the active layer prior to entry into the optical fiber 7.

The base unit 1 can comprise a power supply unit having a current supply for the laser diodes and control electronics in addition to the laser diodes 6 and the heat sink 5 shown. The individual laser diodes 6 can be connected in series, with means in particular being able to be provided for the low-impedance bypass of a failed laser diode 6, in order to ensure smooth operation of the apparatus even in the case of individual laser diodes failing. In the case of this series connection of the individual laser diodes 6, substantially lower currents occur than in the case of laser diode bars. As a result of this, electrical lines with smaller cross sections and simpler electronic circuits can be used.

Furthermore, the base unit 1 can also house the cooling supply for the laser diodes 6, which in particular is formed as an air cooling system or a cooling system having peltier elements.

The optical fibers 7 emanating from the individual laser diodes 6 are combined in a bundle and are a part of the flexible cable 3 which connects the base unit 1 to the portable unit 2. Furthermore, electrical signaling lines, for example for switching or controlling the laser diodes 6, can be contained in the cable 3. In addition, the cable 3 can comprise one or more optical fibers for guiding visible pilot radiation. This pilot radiation can emanate from a laser diode or light-emitting diode provided in the base unit which emits light in the visible range of the spectrum. The pilot radiation can make it clear to the user which path the laser radiation takes after being emitted from the portable unit 2.

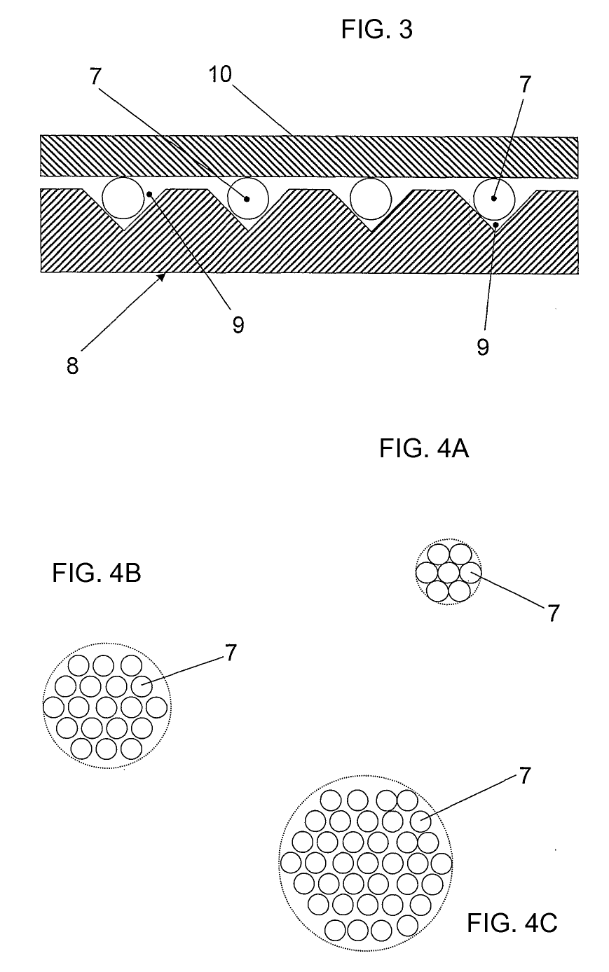

FIG. 3 shows a detail of an exemplary embodiment of a portable unit 2. In particular, this exemplary embodiment comprises a holding part 8 with a plurality of V-shaped grooves 9. One of the optical fibers 7 is arranged in one of each of the grooves 9. The optical fibers 7 are held in the grooves 9 by a slab 10 which bears against the optical fibers 7 on the side facing away from the grooves 9 and which is connected to the holding part 8, for example. The portable unit 2 can comprise a protective screen which is transparent to the laser radiation 4 and which can protect the ends of the optical fibers 7 from external influences. In particular, the portable unit 2 can be hermetically sealed against moisture and the like.

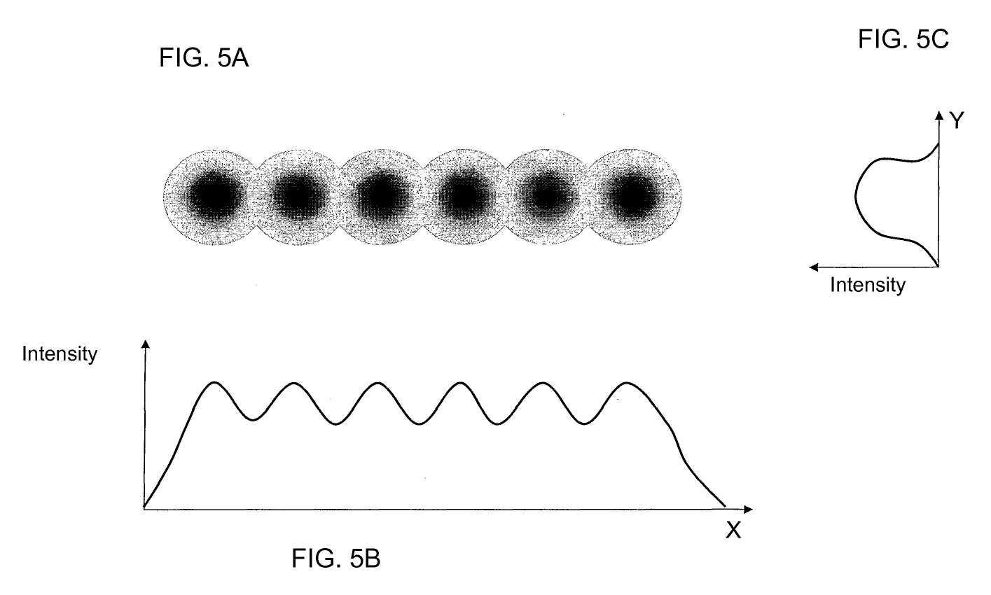

The distance of the bottom ends of the grooves 9 from one another can be between 0.5 mm and 5 mm, in particular approximately 1 mm. The distance of the axes of the optical fibers 7 from one another can thus also be approximately 1 mm. The laser light emitted from the individual optical fibers 7 already overlaps with one another shortly after the end of the optical fibers 7. FIG. 5A illustrates the two-dimensional intensity distribution of the overlapped laser radiation at a distance of 3 mm after the end of the optical fibers 7. In this case, the darker regions correspond to a higher intensity than the lighter regions.

In FIG. 5B and FIG. 5C, the intensity of the laser radiation is plotted in each case against a spatial coordinate X or Y, with the directions X and Y being orthogonal to one another. FIG. 5B clearly shows that the intensity differences between the darker points in FIG. 5A, which can be assigned to the cores of the individual optical fibers 7, and the lighter transitional or overlapping areas are present but not very pronounced. As a result of this, a sufficient homogeneity of the linearly overlapping laser radiation for the shaving of human hair can be ensured at a working distance of 2 mm from the emission location of the laser radiation from the portable unit 2.

The mechanical tolerance requirements for the holding part 8 are very low, since a small change in the distance of the optical fibers 7 from one another only influences the intensity distribution of the overlapped laser radiation in an unsubstantial manner. Correspondingly, the portable unit 2 is as insusceptible as possible to external mechanical or thermal influences on account of the large mechanical tolerances of the holding part 8. This robustness of the portable unit 2 is increased by the protective screen and the hermetic sealing of the portable unit 2.

FIG. 4A illustrates the exemplary arrangement of 7 optical fibers 7, FIG. 4B illustrates the exemplary arrangement of 19 optical fibers 7 and FIG. 4C illustrates the exemplary arrangement of 37 optical fibers 7 in a bundle of optical fibers. The mentioned numbers of optical fibers 7 in each case enable a very compact organization of the optical waveguides 7 in the bundle.

1. A laser shaving apparatus, comprising:
a base unit having at least one laser light source generating laser radiation;
a portable unit movable by a user into an area of hair to be cut;
a transmission device connected between said base unit and said portable unit for transmitting the laser radiation from said at least one laser light source of said base unit to said portable unit, said transmission device including a plurality of optical fibers for transmitting the laser radiation; and
said optical fibers having ends disposed in said portable unit, said ends being arranged to enable the laser radiation emitted therefrom to overlap at least partially and to form an elongated beam cross section in an overlapped state.

2. The apparatus according to claim 1, wherein the beam cross section of the laser radiation is substantially linear in the overlapped state.

3. The apparatus according to claim 1, wherein said ends of said optical fibers are disposed substantially in a row next to one another in said portable unit.

4. The apparatus according to claim 1, wherein said at least one laser light source is one of a plurality of laser light sources.

5. The apparatus according to claim 4, wherein said laser light sources are a plurality of individual laser diodes.

6. The apparatus according to claim 4, wherein said laser light sources are a plurality of individual emitters of a laser diode bar.

7. The apparatus according to claim 4, wherein each one of said laser light sources is assigned exactly one respective optical fiber.

8. The apparatus according to claim 1, wherein said optical fibers are disposed in front of said at least one laser light source to enable the laser light emitted from said at least one laser light source to pass directly into said optical fibers.

9. The apparatus according to claim 8, wherein the laser light does not pass through optical means between said at least one laser light source and said optical fibers.

10. The apparatus according to claim 1, wherein said portable unit is configured to cause the laser radiation emitted from said optical fibers to passes directly into the work area used for shaving.

11. The apparatus according to claim 10, wherein the laser light does not pass through optical means upon issuing from said ends of said optical fibers.

12. The apparatus according to claim 1, wherein said transmission device is a flexible cable having said plurality of optical fibers densely packed therein.

13. The apparatus according to claim 1, wherein said transmission device comprises 7 optical fibers.

14. The apparatus according to claim 1, wherein said transmission device comprises 19 optical fibers.

15. The apparatus according to claim 1, wherein said transmission device comprises 37 optical fibers.

16. The apparatus according to claim 1, wherein said flexible cable includes at least one of an electrical signaling line and/or at least one optical fiber for guiding visible pilot radiation.

17. The apparatus according to claim 1, wherein said portable unit is configured to emit the laser radiation and to shave off hair outside said portable unit.

18. The apparatus according to claim 1, said portable unit is configured to allow hair to partially penetrate into said portable unit and to shave off the hair within said portable unit.

19. A disinfection method, which comprises:
providing an apparatus according to claim 1; and
irradiating an object with the laser radiation for disinfecting the object.

20. A method of processing plastics, which comprises:
providing an apparatus according to claim 1; and
irradiating a plastics object for processing the plastics.
